(19) United States
(12) Patent Application Publication
(10) Pub. No.: US 2020/0210895 A1
(43) Pub. Date: Jul. 2, 2020

(54) TIME SERIES DATA PROCESSING DEVICE AND OPERATING METHOD THEREOF

(71) Applicant: ELECTRONICS AND TELECOMMUNICATIONS RESEARCH INSTITUTE, Daejeon (KR)

(72) Inventors: Youngwoong HAN, Daejeon (KR); Hwin-Dol PARK, Daejeon (KR); Jae-Hun CHOI, Daejeon (KR)

(21) Appl. No.: 16/694,921

(22) Filed: Nov. 25, 2019

(30) Foreign Application Priority Data
Dec. 31, 2018 (KR) 10-2018-0173917

(51) Int. Cl. G06N 20/00; G16H 50/20

(52) U.S. Cl. CPC G06N 20/00; G16H 50/20

(57) ABSTRACT

The time series data processing device according to an embodiment of the inventive concept includes a preprocessor, a learner, and a predictor. The preprocessor preprocesses time series data to generate interval data, interpolation data, and masking data. The learner generates a weight value group of a prediction model that generates a feature weight value and a time series weight value, based on the interval data, the interpolation data, and the masking data. The feature weight value depends on a time and a feature of the time series data and the time series weight value depends on a time flow of the time series data. The predictor generates a feature weight value and a time series weight value, based on the weight value group, and generates a prediction result, based on the feature weight value and time series weight value.

### CROSS-REFERENCE TO RELATED APPLICATIONS

This U.S. non-provisional patent application claims priority under 35 U.S.C. § 119 of Korean Patent Application No. 10-2018-0173917, filed on Dec. 31, 2018, the entire contents of which are hereby incorporated by reference.

### BACKGROUND

Embodiments of the inventive concept relate to processing of time series data, and more particularly, to a time series data processing device for learning or using a prediction model and a method of operating the same.

The development of various technologies including medical technology improves the standard of living of human beings and increases the life span of human beings. However, according to the development of technologies, lifestyle changes and poor eating habits are causing various diseases. To lead a healthy life, there is being raised a demand for predicting future health conditions in addition to treating current diseases. Accordingly, solutions of predicting the health conditions of a future time point are being proposed by analyzing a trend of time series medical data over time.

With the development of industrial technology and information and communication technology, a considerable amount of information and data are generated. In recent years, technologies for providing various services that are obtained by learning electronic devices such as computers, using such numerous information and data, such as an artificial intelligence have emerged. In particular, to predict future health conditions, solutions of constructing a prediction model using various time series medical data has been proposed. For example, time series medical data differs from data collected in other fields in that it has irregular time intervals, and complex and non-specific features. Thus, there is a need to effectively process and analyze time series medical data to predict future health conditions.

### SUMMARY

Embodiments of the inventive concept provide a time series data processing device and a method of operating the same, which improves an accuracy and a reliability of a prediction result by correcting an irregular time interval and a missing value of the time series data.

According to an exemplary embodiment, a time series data processing device includes a preprocessor and a learner. The preprocessor generates interval data, based on a time interval of time series data, adds an interpolation value to a missing value of the time series data to generate interpolation data, and generates masking data for distinguishing the missing value. The learner generates a weight value group of a prediction model that generates a feature weight value depending on a time and a feature of the time series data and a time series weight value depending on a time flow of the time series data, based on the interval data, the interpolation data, and the masking data. The weight value group includes a first parameter for generating the feature weight value and a second parameter for generating the time series weight value.

In an exemplary embodiment, the learner may include a feature learner, a time series learner, and a weight value controller. The feature learner may calculate the feature weight value, based on the masking data, the interval data, the interpolation data, and the first parameter, and generate a first learning result, based on the feature weight value. The time series learner may calculate the time series weight value, based on the first learning result and the second parameter, and generate a second learning result, based on the time series weight value. The weight value controller may adjust the first parameter or the second parameter, based on the first learning result or the second learning result.

In an exemplary embodiment, the feature learner may include a missing value processor to generate first correction data of the interpolation data, based on the masking data, a time processor to generate second correction data of the interpolation data, based on the interval data, a feature weight value calculator to calculate the feature weight value, based on the first parameter, the first correction data, and the second correction data, and a feature weight value applicator to apply the feature weight value to the interpolation data. In an exemplary embodiment, the time series learner may include a time series weight value calculator to calculate the time series weight value, based on the first learning result and the second parameter, and a time series weight value applicator to apply the time series weight value to the first learning result.

In an exemplary embodiment, the learner may include a feature learner, a time series learner, and a weight value controller. The feature learner may calculate the feature weight value, based on the masking data, the interpolation data, and the first parameter, and generate a first learning result, based on the feature weight value. The time series learner may calculate the time series weight value, based on the interval data, the first learning result, and the second parameter, and generate a second learning result, based on the time series weight value. The weight value controller may adjust the first parameter or the second parameter, based on the first learning result or the second learning result.

In an exemplary embodiment, the feature learner may include a missing value processor to generate correction data of the interpolation data, based on the masking data, a feature weight value calculator configured to calculate the feature weight value, based on the first parameter and the correction data, and a feature weight value applicator to apply the feature weight value to the interpolation data. In an exemplary embodiment, the time series learner may include a time processor to generate correction data of the first learning result, based on the interval data, a time series weight value calculator to calculate the time series weight value, based on the second parameter and the correction data, and a time series weight value applicator to apply the time series weight value to the first learning result.

In an exemplary embodiment, the learner may include a feature learner, a time series learner, an integrated weight value applicator, and a weight value controller. The feature learner may calculate the feature weight value, based on the masking data, the interpolation data, and the first parameter. The time series learner may calculate the time series weight value, based on the interval data, the interpolation data, and the second parameter. The integrated weight value applicator may generate a learning result, based on the feature weight value and the time series weight value. The weight value controller may adjust the first parameter or the second parameter, based on the learning result.

According to an exemplary embodiment, a time series data processing device includes a preprocessor and a predictor. The preprocessor generates interval data, based on a time interval of time series data, adds an interpolation value to a missing value of the time series data to generate interpolation data, and generates masking data for distinguishing the missing value. The predictor generates a feature weight value depending on a time and a feature of the time series data and a time series weight value depending on a time flow of the time series data, based on the interval data, the interpolation data, and the masking data. The predictor generates a prediction result, based on the feature weight value and the time series weight value.

In an exemplary embodiment, the predictor may include a feature predictor, a time series predictor, and a result generator. The feature predictor may generate a first result, based on the feature weight value. The time series predictor may generate a second result, based on the time series weight value. The result generator may calculate the prediction result corresponding to a prediction time, based on the second result.

In an exemplary embodiment, the feature predictor may include a missing value processor to encode the interpolation data, based on the masking data, a time processor to model the interval data, a feature weight value calculator to generate feature analysis data, based on the encoded interpolation data and to generate the feature weight value, based on the feature analysis data and the modeled interval data. The feature weight value applicator may apply the feature weight value to the feature analysis data to generate the first result.

In an exemplary embodiment, the feature predictor may include a missing value processor to merge the masking data and the interpolation data, a time processor to model the interval data, a feature weight value calculator to generate feature analysis data, based on the merged data, and generate the feature weight value, based on the feature analysis data and the modeled interval data, and a feature weight value applicator to apply the feature weight value to the feature analysis data to generate the first result.

In an exemplary embodiment, the feature predictor may include a missing value processor to model the masking data, a time processor to model the interval data, a feature weight value calculator to generate feature analysis data, based on the interpolation data, and generate the feature weight value, based on the modeled masking data, the modeled interval data, and the feature analysis data, and a feature weight value applicator to apply the feature weight value to the feature analysis data to generate the first result.

In an exemplary embodiment, the feature predictor may include a missing value processor to model the masking data, a time processor to merge the interval data and the interpolation data, a feature weight value calculator to generate feature analysis data, based on the merged data, and generate the feature weight value, based on the feature analysis data and the modeled masking data, and a feature weight value applicator to apply the feature weight value to the feature analysis data to generate the first result.

In an exemplary embodiment, the time series predictor may include a time series weight value calculator to generate time series analysis data, based on the first result, and generate the time series weight value, based on the time series analysis data, and a time series weight value applicator to apply the time series weight value to the first result or the time series analysis data.

According to an exemplary embodiment, a method of operating a time series data processing device, includes generating interpolation data, generating interval data, generating masking data, generating a feature weight value depending on a time and a feature of the time series data, based on the interpolation data, the interval data, and the masking data, generating a first result, based on the feature weight value, generating a time series weight value depending on a time flow of the time series data, based on the first result, and generating a second result, based on the time series weight value.

In an exemplary embodiment, the method may further includes adjusting a parameter for generating the feature weight value or the time series weight value, based on the second result. In an exemplary embodiment, the method may further includes calculating a prediction result corresponding to a prediction time, based on the second result.

### BRIEF DESCRIPTION OF THE FIGURES

The above and other objects and features of the inventive concept will become apparent by describing in detail exemplary embodiments thereof with reference to the accompanying drawings.

FIG. 1 is a block diagram illustrating a time series data processing device according to an embodiment of the inventive concept.

FIG. 2 is a graph describing time series irregularities and missing values of time series data described in FIG. 1.

FIG. 3 is an exemplary block diagram illustrating a preprocessor of FIG. 1.

FIG. 4 is an exemplary block diagram illustrating a learner of FIG. 1.

FIG. 5 is an exemplary block diagram illustrating a predictor of FIG. 1.

FIGS. 6 to 9 are diagrams illustrating in detail a predictor of FIG. 5.

FIGS. 10 and 11 are exemplary block diagrams illustrating a learner or a predictor of FIG. 1.

FIG. 12 is a diagram illustrating a health condition prediction system to which a time series data processing device of FIG. 1 is applied.

FIG. 13 is an exemplary block diagram illustrating a time series data processing device of FIG. 1 or FIG. 12.

### DETAILED DESCRIPTION

Embodiments of the inventive concept will be described below in more detail with reference to the accompanying drawings. In the following descriptions, details such as detailed configurations and structures are provided merely to assist in an overall understanding of embodiments of the inventive concept. Modifications of the embodiments described herein can be made by those skilled in the art without departing from the spirit and scope of the inventive concept. Furthermore, descriptions of well-known functions and structures are omitted for clarity and brevity. The terms used in this specification are defined in consideration of the functions of the inventive concept and are not limited to specific functions. Definitions of terms may be determined based on the description in the detailed description.

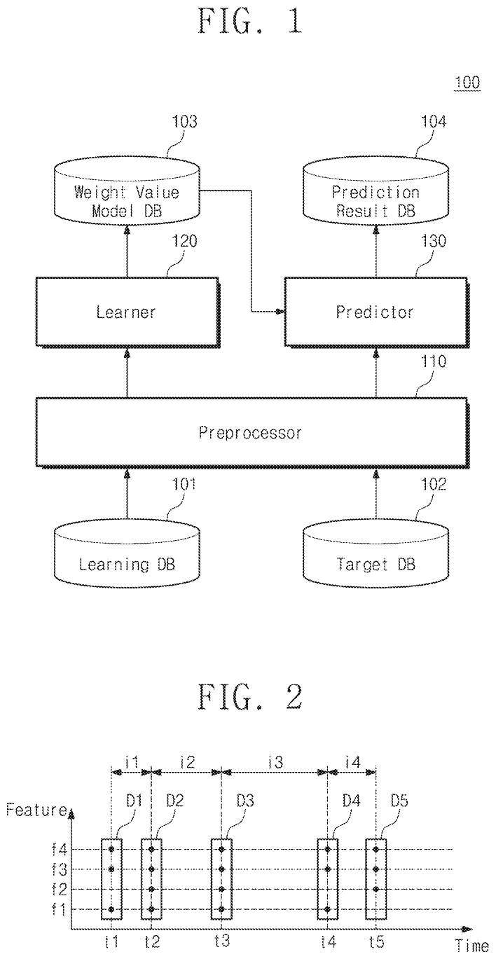

FIG. 1 is a block diagram illustrating a time series data processing device according to an embodiment of the inventive concept. A time series data processing device 100 of FIG. 1 may be understood as an exemplary configuration for preprocessing time series data, learning a prediction model by analyzing the preprocessed time series data, or generating a prediction result. Referring to FIG. 1, the time series data processing device 100 includes a preprocessor 110, a learner 120, and a predictor 130.

The preprocessor 110, the learner 120, and the predictor 130 may be implemented in hardware, firmware, software, or a combination thereof. As an example, software (or firmware) may be loaded into a memory (not illustrated) that is included in the time series data processing device 100 and may executed by a processor (not illustrated). For example, the preprocessor 110, the learner 120, and the predictor 130 may be implemented in hardware such as a dedicated logic circuit such as a field programmable gate array (FPGA) or an application specific integrated circuit (ASIC).

The preprocessor 110 may preprocess the time series data. The time series data may be a data set with a temporal order, recorded over time. The time series data may include at least one feature corresponding to each of the plurality of times that are listed in time series. As an example, the time series data may include time series medical data that represent a state of health of a user generated by a diagnosis, treatment, or dosage prescription in a medical institution, such as an electronic medical record (EMR). For clarity of explanation, although the time series medical data has been described as an example, but the type of time series data is not limited thereto. The time series data may be generated in various fields such as entertainment, retail, and smart management.

The preprocessor 110 may preprocess the time series data to correct a time series irregularity, a missing value, a type difference between features, and the like, of the time series data. The time series irregularity means that a time interval between a plurality of times is not regular. The missing value means a feature that is missing or not present at a certain time of the plurality of features. The type difference between the features means that criteria for generating a value are different for each feature. The preprocessor 110 may preprocess the time series data such that the time series irregularity is applied in the time series data, the missing value is interpolated, and the type between the features is matched. Details thereof will be described later.

The learner 120 may learn a prediction model, based on the preprocessed time series data. The prediction model may include a time series analysis model for analyzing the preprocessed time series data to calculate a prediction result of a future. As an example, the prediction model may be built through an artificial neural network or deep learning machine learning. To this end, the time series data processing device 100 may receive the time series data for learning from a learning database 101. The learning database 101 may be implemented in a server or a storage medium outside or inside the time series data processing device 100. In the learning database 101, data may be managed in a time series, grouped, and stored. The preprocessor 110 may preprocess the time series data received from the learning database 101 and provide it to the learner 120.

The learner 120 may analyze the preprocessed time series data to generate a weight value group of the prediction model. The learner 120 may generate a prediction result through analysis of the time series data, and adjust the weight value group of the prediction model such that the generated prediction result has an expected value. The weight value group may be a neural network structure of the prediction model or a set of all parameters included in the neural network. The weight value group and the prediction model may be stored in a weight value model database 103. The weight value model database 103 may be implemented in a server or a storage medium outside or inside the time series data processing device 100. The weight value group and the prediction model may be managed and stored in the weight value model database 103.

The predictor 130 may generate the prediction result by analyzing the preprocessed time series data. The prediction result may be a result corresponding to a prediction time such as a specific point in time in the future. To this end, the time series data processing device 100 may receive the time series data for prediction from a target database 102. The target database 102 may be implemented in a server or a storage medium outside or inside the time series data processing device 100. In the target database 102, data may be managed in a time series, grouped and stored. The preprocessor 110 may preprocess the time series data received from the target database 102 and provide it to the predictor 130.

The predictor 130 may analyze the preprocessed time series data, based on the prediction model learned from the learner 120 and the weight value group. To this end, the predictor 130 may receive the weight value group and the prediction model from the weight value model database 103. The predictor 130 may calculate the prediction result by analyzing trends of the time series in the preprocessed time series data. The prediction result may be stored in a prediction result database 104. The prediction result database 104 may be implemented in a server or a storage medium outside or inside the time series data processing device 100.

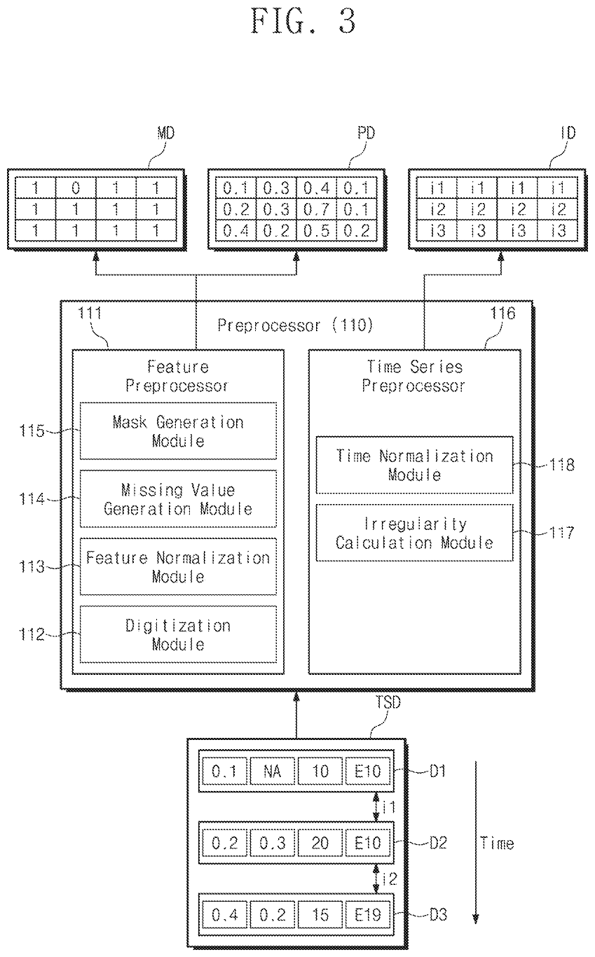

FIG. 2 is a graph describing time series irregularities and missing values of time series data described in FIG. 1. A horizontal axis represents a time and a vertical axis represents features in FIG. 2. Referring to FIG. 2, it is assumed that time series data includes first to fifth data D1 to D5 listed in a time series. It is assumed that the time series data includes first to fourth features f1 to f4. For convenience of explanation, it is assumed that the time series data of FIG. 2 includes medical data.

The time series data may be organized in two dimensions including a time and a feature. That is, the time series data may include a plurality of features f1 to f4 corresponding to a plurality of times t1 to t5. By analyzing such time series data, the prediction result corresponding to a future time point may be calculated. To improve an accuracy and reliability of the prediction result, the prediction model that considers both the time and the feature may be required. The time series data processing device 100 of FIG. 1 may apply both the time and the feature of the time series data to perform learning and prediction. Such details will be described later.

The time series data may have the missing value. For example, the first data D1 and the fourth data D4 may not include the second feature f2, and the fifth data D5 may not include the first feature f1. These features may be defined as missing values. The features of the time series data may be generated, based on the diagnosis, treatment, or dosage prescription in the medical institution. Since medical institutions do not always perform the same tests and the like, the missing value may occur in the time series data. When the time series data is analyzed, the missing value decreases the accuracy and reliability of the prediction result or the learning result. The time series data processing device 100 of FIG. 1 may perform learning and prediction in consideration of the missing value of the time series data. Such details will be described later.

The time series data may have irregular time intervals. The first to fifth data D1 to D5 may be generated, measured, or recorded at the first to fifth times t1 to t5, respectively. For example, the first to fifth times t1 to t5 may be times at which the diagnosis, treatment, or dosage prescription is performed at the medical institution. As illustrated in FIG. 2, the first to fourth time intervals i1 to i4 among the first to fifth times t1 to t5 may be irregular. The reason why the first to fourth time intervals i1 to i4 are irregular is that a visit of the medical institution is not constant. Typical time series analysis assumes that time intervals are constant, such as data collected at constant time through a sensor. Such analysis may not consider irregular time intervals. The time series data processing device 100 of FIG. 1 may perform the learning and the prediction by applying the irregular time interval. Such details will be described later.

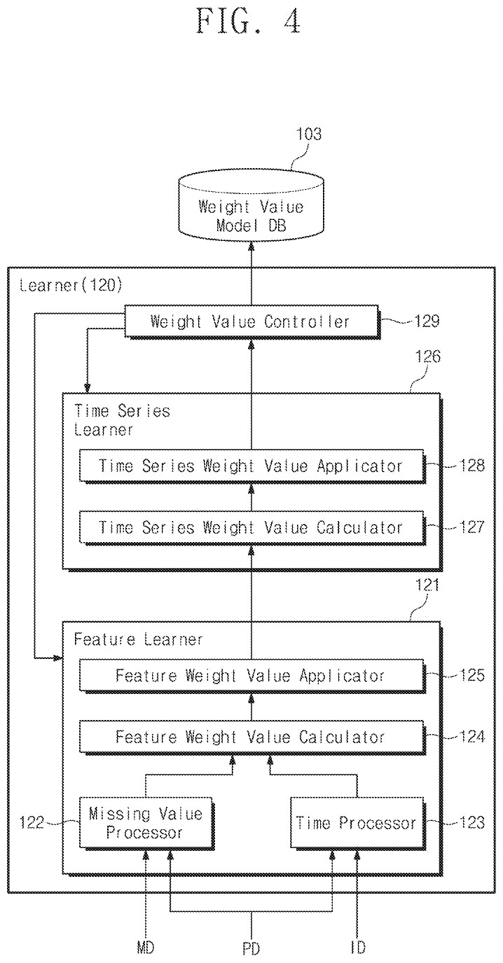

FIG. 3 is an exemplary block diagram illustrating a preprocessor of FIG. 1. The block diagram of FIG. 3 will be understood as an exemplary configuration for preprocessing the time series data (TSD), in consideration of the complexity of the time and the feature, the presence of the missing value, and the irregular time interval, as described in FIG. 2. Referring to FIG. 3, the preprocessor 110 may include a feature preprocessor 111 and a time series preprocessor 116. As described in FIG. 1, the feature preprocessor 111 and the time series preprocessor 116 may be implemented in hardware, firmware, software, or a combination thereof.

The feature preprocessor 111 and the time series preprocessor 116 receive the time series data TSD. The time series data TSD may be data for learning the prediction model or data for calculating the prediction result through the learned prediction model. In exemplary embodiments, the time series data TSD includes first to third data D1 to D3, and correspond to the first to third data D1 to D3 of FIG. 2. Each of the first to third data D1 to D3 may include first to fourth features. As illustrated in FIG. 2, the first data D1 does not include the second feature f2.

The feature preprocessor 111 may preprocess the time series data TSD to generate interpolation data PD. The interpolation data PD may include features of the time series data TSD that are converted to have the same type. The interpolation data PD may have the same number of times and features as the time series data TSD. The interpolation data PD may be time series data obtained by interpolating the missing value. When the features of the time series data (TSD) have the same type and the missing value is interpolated, the time series analysis by the learner 120 or the predictor 130 of FIG. 1 may be relatively easy. To generate the interpolation data PD, a digitization module 112, a feature normalization module 113, and a missing value generation module 114 may be implemented in the feature preprocessor 111.

The feature preprocessor 111 may generate the masking data MD by preprocessing the time series data TSD. The masking data MD may be data for distinguishing the missing values and real values of the time series data TSD. The masking data MD may have the same number of the times and the features as the time series data TSD. The masking data MD may be generated during the time series analysis such that the missing value is not treated with the same importance as the real value. To generate the masking data MD, a mask generation module 115 may be implemented in the feature preprocessor 111.

The digitization module 112 may convert non-numeric features of types in the time series data TSD into numeric types. The non-numeric types may include code types or categorical types (e.g., −, +, ++, etc.). For example, the EMR data may have a prescribed data type, depending on particular disease, prescription, or test, but may have a mix type of numerical and non-numeric types. For example, the fourth feature of each of the first to third data D1 to D3 has values E10, E10, and E19 which are not a numerical value. The digitization module 112 may convert the fourth features E10, E10, and E19 of the time series data TSD into numerical types such as the fourth features (0.1, 0.1, and 0.2) of the interpolation data PD. As an example, the digitization module 112 may digitize the features in an embedding manner such as Word2Vec.

The feature normalization module 113 may convert numeric values of the time series data TSD into values of a reference range. For example, the reference range may include a value between 0 to 1, or between −1 to 1. The time series data TSD may have the numerical values in an independent range, depending on the feature. For example, a third feature of each of the first to third data D1 to D3 has numerical values 10, 20, and 15 outside the reference range. The feature normalization module 113 may normalize the third features 10, 20, and 15 of the time series data TSD to the reference range such as the third features (0.4, 0.7, and 0.5) of the interpolation data PD.

The missing value generation module 114 may add the interpolation value to the missing value of the time series data TSD. The interpolation value may have a preset value or may be generated based on different values of the time series data TSD. For example, the interpolation value may have a zero, an intermediate value of features of another time, an average value, or a feature value of an adjacent time. For example, the second feature of the first data D1 has the missing value. The missing value generation module 114 may set an interpolation value as 0.3, which is a second feature value of the second data D2 that is temporally adjacent to the first data D1.

The mask generation module 115 generates the masking data MD, based on the missing value. The mask generation module 115 may generate the masking data MD by differently setting a value corresponding to a missing value and a value (real value) corresponding to the different values. For example, the value corresponding to the missing value may be 0 and the value corresponding to the real value may be 1.

The time series preprocessor 116 may preprocess the time series data TSD to generate interval data ID. The interval data ID may include time interval information between data of adjacent times of the time series data TSD. The interval data ID may have the same number of values as the time series data TSD in the time dimension. The interval data ID may have the same number of values as the time series data TSD or one value in the feature dimension. In exemplary embodiments, the first data D1 and the second data D2 may have a first time interval i1, and the second data D2 and the third data D3 may have a second time interval i2. The interval data ID may be generated such that time series irregularities are considered, in the time series analysis. To generate the interval data ID, an irregularity calculation module 117 and a time normalization module 118 may be implemented in the time series preprocessor 116.

The irregularity calculation module 117 may calculate the irregularity of the time series data TSD. The irregularity calculation module 117 may calculate the time interval, based on a time difference between data corresponding to the certain time and data corresponding to the adjacent time. For example, the first data D1 and the second data D2 may have the first time interval i1, and the second data D2 and the third data D3 may have the second time interval i2. Each of the first time interval i1 and the second time interval i2 may correspond to the first data D1 and the second data D2. As an example, the first and second time intervals i1, i2 may be directly applied to the interval data ID. Alternatively, when an ideal reference time interval is set, a difference between the reference time interval and the first or second time intervals i1 and i2 may be applied to the interval data ID.

The time normalization module 118 may normalize the irregularity calculated from the irregularity calculation module 117. The time normalization module 118 may convert the numerical value calculated from the irregularity calculation module 117 into a value of the reference range. For example, the reference range may include a value between 0 to 1, or between −1 to 1. The time digitized by year, month, day, etc. may be out of the reference range, and the time normalization module 118 may normalize the time to the reference range.

FIG. 4 is an exemplary block diagram illustrating a learner of FIG. 1. The block diagram of FIG. 4 will be understood as an exemplary configuration for learning the prediction model and determining the weight value group, based on the preprocessed time series data. Referring to FIG. 4, the learner 120 may include a feature learner 121, a time series learner 126, and a weight value controller 129. As described in FIG. 1, the feature learner 121, the time series learner 126, and the weight value controller 129 may be implemented in hardware, firmware, software, or a combination thereof.

The feature learner 121 analyzes the time and the feature of the time series data, based on interpolation data PD, masking data MD, and interval data ID which are generated from the preprocessor 110 of FIG. 3. The feature learner 121 may learn at least a portion of the prediction model to generate parameters for generating the feature weight value. These parameters (feature parameters) are included in the weight value group. The feature weight value depends on the time and the feature of the time series data.

The feature weight value may include a weight value of each of the plurality of features corresponding to the certain time. That is, the feature weight value may be understood as an index for determining the importance of the values included in the time series data that are calculated based on the feature parameter. To this end, a missing value processor 122, a time processor 123, a feature weight value calculator 124, and a feature weight value applicator 125 may be implemented in the feature learner 121.

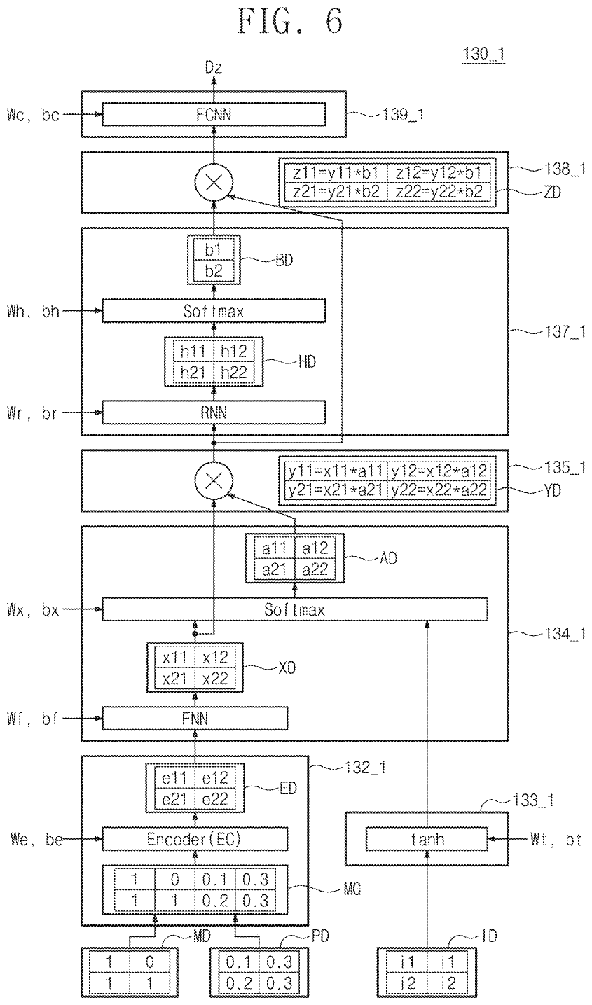

The missing value processor 122 may generate first correction data for correcting an interpolation value of the interpolation data PD, based on the masking data MD. Alternatively, the missing value processor 122 may generate the first correction data by applying the masking data MD to the interpolation data PD. As described above, the interpolation value may be a value obtained by substituting the missing value with a different numeric value. The learner 120 may not know whether the values that are included in the interpolation data PD are randomly assigned interpolation values or real values. Therefore, the missing value processor 122 may generate the first correction data for adjusting the importance of the interpolation value by using the masking data MD. Operations of the missing value processor 122 will be described later with reference to FIGS. 6 to 9.

The time processor 123 may generate second correction data for correcting the irregularity of the time interval of the interpolation data PD, based on the interval data ID. Alternatively, the time processor 123 may generate the second correction data by applying the interval data ID to the interpolation data PD. The time processor 123 may generate the second correction data for adjusting the importance of each of the plurality of times corresponding to the interpolation data PD, using the interval data ID. That is, the features corresponding to the certain time may be corrected with the same importance by the second correction data. Operations of the time processor 123 will be described in detail below with reference to FIGS. 6 to 9.

The feature weight value calculator 124 may calculate the feature weight value corresponding to the features and the times of the interpolation data PD, based on the first correction data and the second correction data. The feature weight value may have the same number of values as the interpolation data PD in the time dimension and the feature dimension. The feature weight value calculator 124 may apply the importance of each of the times and the importance of the interpolation value to the feature weight value. In an example, the feature weight value calculator 124 may generate the feature weight value by using an attention mechanism such that the prediction result pays attention to a specified feature. Operations of the feature weight value calculator 124 will be described below in detail with reference to FIGS. 6 to 9.

The feature weight value applicator 125 may apply the feature weight value that is calculated from the feature weight value calculator 124, to the interpolation data PD. As a result of the application, the feature weight value applicator 125 may generate a first learning result in which the complexity of the time and the feature is applied in the interpolation data PD. For example, the feature weight value applicator 125 may multiply the feature weight value corresponding to the certain time and feature by the feature corresponding to the interpolation data PD. However, the inventive concept is not limited thereto, and the feature weight value may be applied to an intermediate result that is obtained by analyzing the interpolation data PD with the first or second correction data instead of the interpolation data PD. Operations of the feature weight value applicator 125 will be described below in detail with reference to FIGS. 6 to 9.

The time series learner 126 analyzes a time flow of the time series data, based on the first learning result that is generated from the feature weight value applicator 125. When the feature learner 121 analyzes values corresponding to the feature and the time of the time series data (herein, the time may mean the certain time point at which the time interval is applied), the time series learner 126 may analyze trends of the data depending on the time flow, or relationship between the prediction time and the certain time. The time series learner 126 may generate parameters for generating time series weight value by learning at least a portion of the prediction model. These parameters (time series parameters) are included in the weight value group.

The time series weight value may include the weight value of each of the plurality of times corresponding to the time flow. That is, the time series weight value may be understood as an index for determining the importance of each of the times of the time series data, which is calculated based on the time series parameter. To this end, a time series weight value calculator 127 and a time series weight value applicator 128 may be implemented in the time series learner 126.

The time series weight value calculator 127 may calculate the time series weight value corresponding to the times of the first learning result that is generated from the feature learner 121. The time series weight value may have the same number of values as the first learning result in the time dimension, but may have one value in the feature dimension. The time series weight value calculator 127 may apply the importance of each of the times corresponding to the prediction time to the time series weight value. In exemplary embodiments, the time series weight value calculator 127 may generate time series weight value by using the attention mechanism such that the prediction result pays attention to a specified time. Operations of the time series weight calculator 127 will be described in detail later with reference to FIGS. 6 to 9.

The time series weight value applicator 128 may apply the time series weight value that is calculated from the time series weight value calculator 127 to the first learning result. As a result of the application, the time series weight value applicator 128 may generate a second learning result in which the irregularity of the time interval and the time series trend are applied. For example, the time series weight value applicator 128 may multiply the time series weight value corresponding to the certain time by the features of the first learning result corresponding to the certain time. However, the inventive concept is not limited thereto, and the time series weight value may be applied to an intermediate result that is obtained by analyzing the first learning result instead of the first learning result. Operations of the time series weight applicator 128 will be described in detail below with reference to FIGS. 6 to 9.

The weight value controller 129 may adjust the feature parameter and the time series parameter, based on the second learning result. The weight value controller 129 may determine whether the second learning result corresponds to a desired real result. The weight value controller 129 may adjust the feature parameter and the time series parameter such that the second learning result reaches the desired real result. Based on the adjusted feature parameter and the adjusted time series parameter, the feature learner 121 and the time series learner 126 may iteratively analyze the preprocessed time series data. These feature parameters and time series parameters may be stored in the weight value model database 103. Unlike illustrated FIG. 4, the weight value controller 129 may further receive the first learning result from the feature learner 121, and adjust the feature parameter, based on the first learning result.

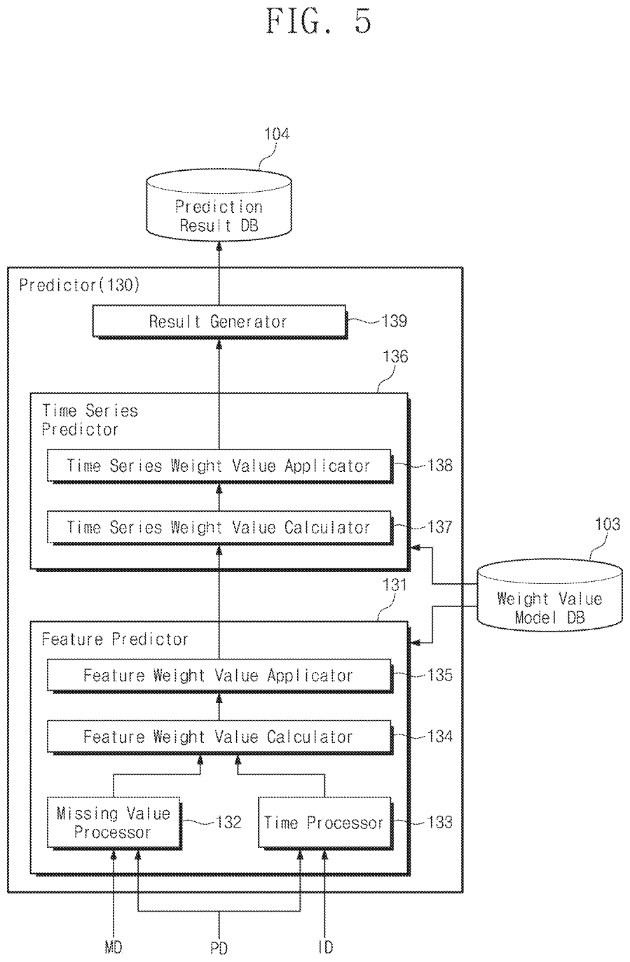

FIG. 5 is an exemplary block diagram illustrating a predictor of FIG. 1. The block diagram of FIG. 5 will be understood as an exemplary configuration for analyzing preprocessed time series data and generating the prediction result, based on the predictive model and weight value group learned by the learner 120 of FIG. 1. Referring to FIG. 5, the predictor 130 may include a feature predictor 131, a time series predictor 136, and a result generator 139. As described in FIG. 1, the feature predictor 131, the time series predictor 136, and the result generator 139 may be implemented in hardware, firmware, software, or a combination thereof.

The feature predictor 131 analyzes the time and the feature of the time series data, based on the interpolation data PD, the masking data MD, and the interval data ID that are generated from the preprocessor 110 of FIG. 3. A missing value processor 132, a time processor 133, a feature weight value calculator 134, and a feature weight value applicator 135 may be implemented in the feature predictor 131 and may be implemented substantially the same as the missing value processor 122, the time processor 123, the feature weight value calculator 124, and the feature weight value applicator 125 in FIG. 4. The feature predictor 131 may analyze the preprocessed time series data, based on the feature parameter provided from the weight value model database 103 and generate a first result.

The time series predictor 136 analyzes the time flow of the time series data, based on the first result that is generated from the feature predictor 131. A time series weight value calculator 137 and a time series weight value applicator 138 may be implemented in the time series predictor 136 and may be implemented substantially the same as the time series weight value calculator 127 and the time series weight value applicator 128 in FIG. 4. The time series predictor 136 may analyze the first result and generate a second result, based on the time series parameter that is provided from the weight value model database 103.

The result generator 139 may calculate the prediction result corresponding to the prediction time, based on the second result that is generated from the time series predictor 136. For example, when the time series data is the medical data, the prediction result may represent conditions of health at a specific time in the future. The prediction result may be stored in the prediction result database 104.

FIGS. 6 to 9 are diagrams illustrating in detail a predictor of FIG. 5. Referring to FIGS. 6 to 9, predictors 130_1 to 130_4 may be implemented as missing value processors 132_1 to 132_4, time processors 133_1 to 133_4, feature weight value calculators 134_1 to 134_4, feature weight value applicators 135_1 to 135_4, and time series weight value calculators 137_1 to 137_4, time series weight value applicators 138_1 to 138_4, and result generators 139_1 to 139_4. Here, the missing value processors 132_1 to 132_4, the time processors 133_1 to 133_4, the feature weight calculators 134_1 to 134_4, and the feature weight applicators 135_1 to 135_4 correspond to the feature predictor 131 of FIG. 5, and the time series weight value calculators 137_1 to 137_4 and the time series weight value applicators 138_1 to 138_4 correspond to the time series predictor 136 of FIG. 5. As described above, since the predictor may be implemented substantially the same as the learner, the predictor structure of FIGS. 6 to 9 may be applied to the learner 120 of FIG. 4.

Referring to FIG. 6, the missing value processor 132_1 may merge the masking data MD and the interpolation data PD to generate merged data MG. The merged data MG may be data obtained by simply arranging values of the masking data MD and the interpolation data PD. That is, the merged data MG may have the same number of values in the time dimension as compared to the masking data MD and the interpolation data PD, and may have twice the number of values in the feature dimension as compared to the masking data MD and the interpolation data PD.

The missing value processor 132_1 may encode the merged data MG to generate encoded data ED. For encoding, the missing value processor 132_1 may include an encoder EC. For example, the encoder EC may be implemented as a one-dimensional (1D) convolutional layer or an auto-encoder. When the encoder is implemented with the 1D convolutional layer, the encoder EC may generate encoding data ED through a kernel that applies the weight value to each of the values of the masking data MD and the values of the interpolation data PD at the same position and adds the applied results. When the encoder is implemented as the auto-encoder, the encoder EC may generate the encoding data ED, based on the encoding function to which the weight value (We) and the bias (be)are applied. The weight value (We) and the bias (be) may be included in the feature parameters described above and may be generated by the learner 120. The encoding data ED may have the same number of values as the value of the masking data MD and the value of the interpolation data PD in the time dimension. The encoding data ED may have the same or different number of values in the feature dimension as the value of the masking data MD and the value of the interpolation data PD. The encoding data ED corresponds to the first correction data described in FIG. 4.

The time processor 133_1 may model the interval data ID. For example, the time processor 133_1 may model the interval data ID by using a nonlinear function such as tan h. In this case, a weight value (Wt) and a bias (bt) may be applied to the corresponding function. For example, the time processor 133_1 may model the interval data ID by calculating equation of tan h (Wt*ID+bt). The weight value (Wt) and the bias (bt) may be included in the feature parameter described above and may be generated by the learner 120. The modeled interval data ID correspond to the second correction data described in FIG. 4.

The feature weight calculator 134_1 may generate the feature weight AD by using an attention mechanism such that the prediction result pays attention to the specified feature. In addition, the feature weight calculator 134_1 may process the modeled interval data together such that the feature weight value AD applies the time interval of the time series data.

In detail, the feature weight value calculator 134_1 may analyze features of the encoding data ED through a feed-forward neural network. The encoding data ED may be correction data that are obtained by applying the importance of the missing value to the interpolation data PD, by the masking data MD. The feed-forward neural network may analyze the encoding data ED, based on the weight value Wf and the bias bf. The weight value Wf and the bias bf may be included in the feature parameter described above and may be generated by the learner 120. The feature weight value calculator 134_1 may analyze the encoding data ED to generate feature analysis data XD. The feature analysis data XD may have the same number of values as the values of the interpolation data PD in the time dimension. The feature analysis data XD may have a number of values that are the same as or different from those of the interpolation data PD in the feature dimension.

The feature weight value calculator 134_1 may calculate the feature weight value AD by applying the feature analysis data XD and the modeled interval data to a softmax function. In this case, a weight value Wx and a bias bx may be applied to the corresponding function. As an example, the feature weight value calculator 134_1 may generate the feature weight value AD by calculating equation of AD=softmax (tan h (Wx*XD+bx)+tan h (Wt*ID+bt)). The weight value Wx and the bias bx may be included in the feature parameter described above and may be generated by the learner 120. As an example, the feature weight value AD may have the same number of values as the feature analysis data XD.

The feature weight value applicator 135_1 may apply the feature weight AD to the feature analysis data XD. As an example, the feature weight value applicator 135_1 may generate a first result YD by multiplying the feature weight value AD by the feature analysis data XD. However, the inventive concept is not limited thereto, and the feature weight value AD may be applied to the interpolation data PD instead of the feature analysis data XD.

The time series weight value calculator 137_1 may generate the time series weight value BD such that the prediction result pays attention to the specified time, by using the attention mechanism. The time series weight value calculator 137_1 may analyze the time flow of the first result YD through a recurrent neural network. The recurrent neural network is a kind of time series analysis algorithm, and may apply data analysis contents of a previous time to the data of a subsequent time. As data having a uniform time interval is input, an analysis accuracy of the recurrent neural network is improved. The first result YD may be a corrected result such as having a uniform time interval, in consideration of the irregularity of the time interval, by the interval data ID. Therefore, the analysis accuracy by the recurrent neural network may be improved.

The time series weight value calculator 137_1 may analyze the first result YD by applying the weight value Wr and the bias br to the recurrent neural network. The weight value Wr and the bias br may be included in the time series parameter described above and may be generated by the learner 120. The time series weight value calculator 137_1 may generate time series analysis data HD by analyzing the first result YD. The time series analysis data HD may have the same number of values as the interpolation data PD in the time dimension. The time series analysis data HD may have the same or different number of values as the interpolation data PD in the feature dimension.

The time series weight value calculator 137_1 may calculate the time series weight value BD, by applying the time series analysis data HD to the softmax function. In this case, a weight value Wh and a bias bh may be applied to the corresponding function. As an example, the time series weight value calculator 137_1 may generate the time series weight value BD by calculating an equation of BD=softmax (tan h (Wh*HD+bh)). The weight value Wh and the bias bh may be included in the time series parameter described above and may be generated by learner 120. The time series weight value BD may have the same number of values as the first result YD in the time dimension. The time series weight value BD may have one value corresponding to each of the plurality of times in the feature dimension.

The time series weight value applicator 138_1 may apply the time series weight value BD to the first result YD. As an example, the time series weight value applicator 138_1 may generate a second result ZD, by multiplying the time series weight value BD by the first result YD. However, the inventive concept is not limited thereto, and the time series weight value BD may be applied to the time series analysis data HD instead of the first result YD.

The result generator 139_1 calculates a prediction result Dz corresponding to the prediction time, based on the second result ZD. The result generator 139_1 may analyze the second result ZD through a fully-connected neural network. The fully-connected neural network may analyze the second result ZD, based on a weight value Wc and a bias bc. The weight value Wc and the bias bc may be included in the weight value group and may be generated by the learner 120. As an example, the prediction result Dz may be a set of features corresponding to a specific time point in the future or a health indicator based on the features.

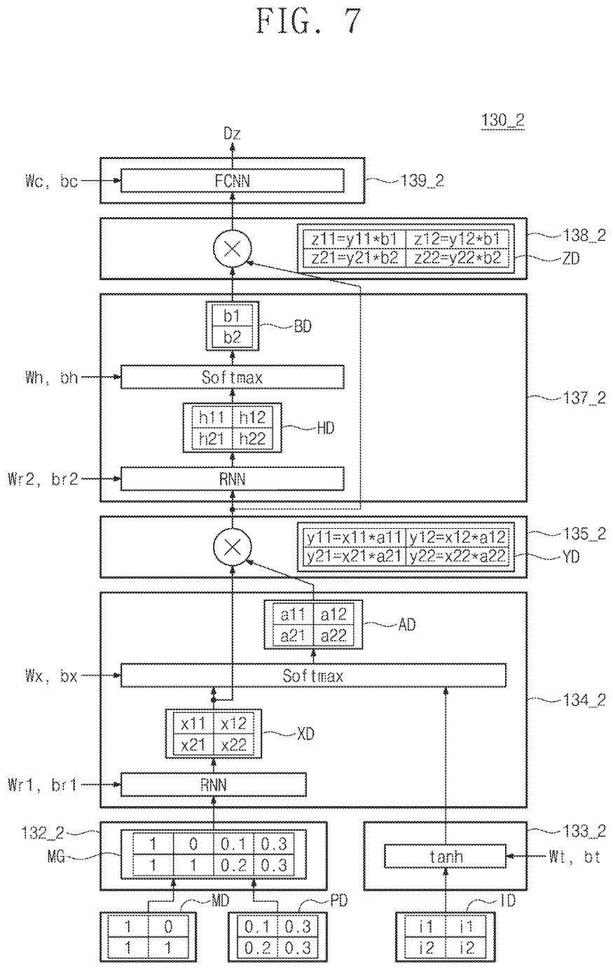

Referring to FIG. 7, a predictor 130_2 may operate substantially the same as the predictor 130_1 of FIG. 6 except for a missing value processor 132_2 and a feature weight value calculator 134_2. Descriptions of components that operate substantially the same will be omitted.

The missing value processor 132_2 may merge the masking data MD and the interpolation data PD to generate merged data MG. Unlike FIG. 6, the missing value processor 132_2 may not post-process the merged data MG. As an example, the feature weight value calculator 134_2 may analyze the merged data MG through the recurrent neural network, instead of the feed-forward neural network. The recurrent neural network may additionally perform a function of encoding the merged data MG. The recurrent neural network may analyze the merged data MG, based on a weight value Wr1 and a bias br1.

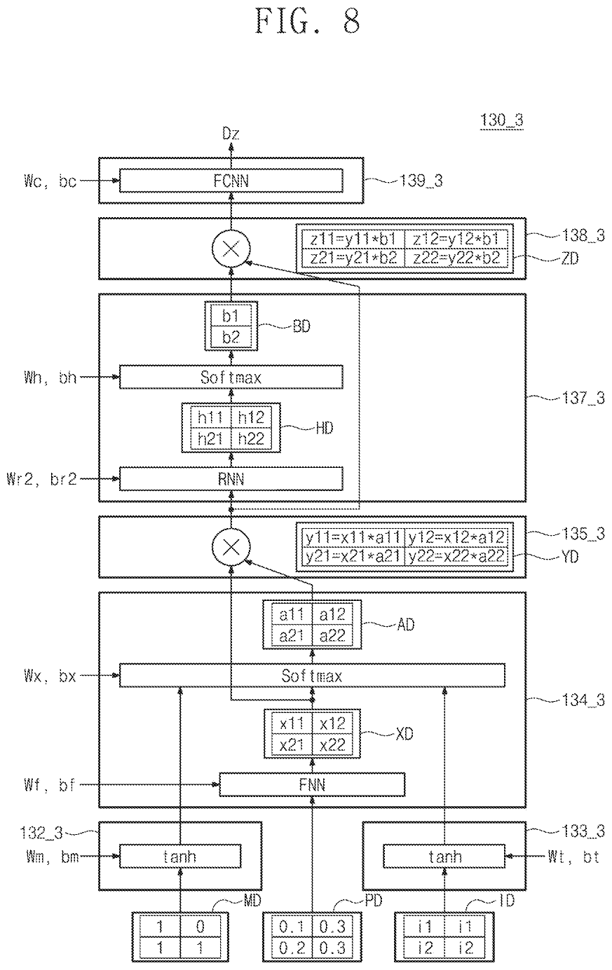

Referring to FIG. 8, a predictor 130_3 may operate substantially the same as the predictor 130_1 of FIG. 6 except for a missing value processor 132_3 and a feature weight value calculator 134_3. Descriptions of components that operate substantially the same will be omitted.

A missing value processor 132_3 may model the masking data MD. For example, the missing value processor 132_3 may model the masking data MD, by using the nonlinear function such as the tan h. In this case, a weight value Wm and a bias bm may be applied to the corresponding function. As an example, the missing value processor 132_3 may model the masking data MD, by calculating an equation of tan h (Wm*MD+bm). The weight value Wm and the bias bm may be included in the feature parameter described above and may be generated by the learner 120.

The feature weight value calculator 134_3 may process the modeled masking data, using the attention mechanism, similar to the modeled interval data. The feature weight value calculator 134_3 may analyze the features of the interpolation data PD and generate the feature analysis data XD through the feed-forward neural network. The feature weight value calculator 134_3 may calculate the feature weight value AD, by applying the feature analysis data XD, the modeled masking data, and the modeled interval data to the softmax function. As an example, the feature weight value calculator 134_3 may generate the feature weight value AD, by calculating an equation of AD=softmax (tan h (Wm*MD+bm)+tan h (Wx*XD+bx)+tan h (Wt*ID+bt)).

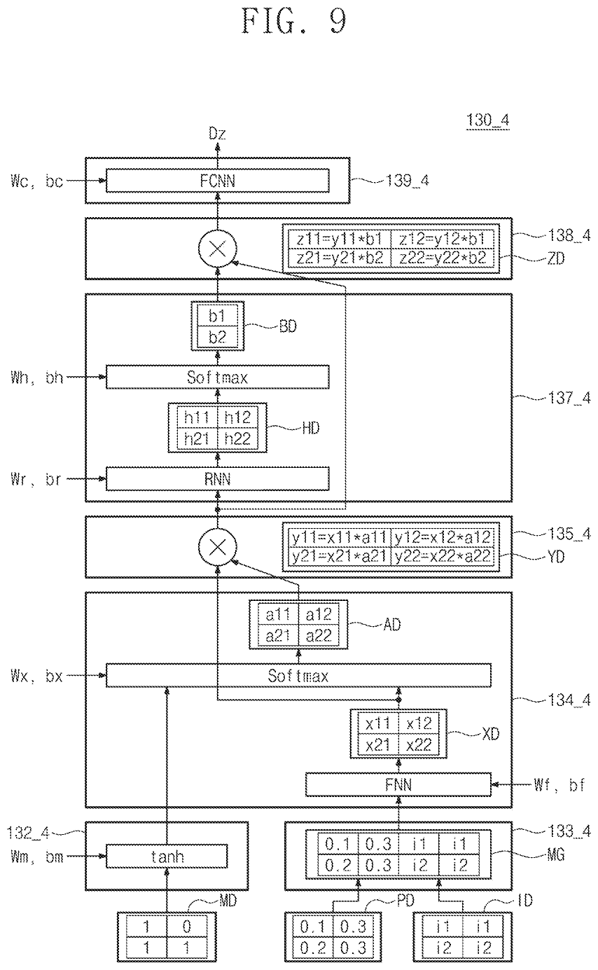

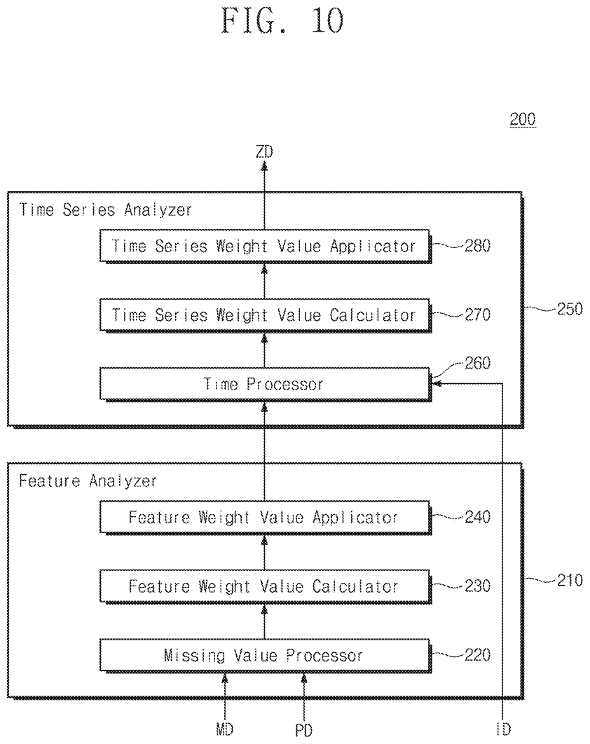

Referring to FIG. 9, a predictor 130_4 may operate substantially the same as the predictor 130_3 of FIG. 8 except for a time processor 133_4 and a feature weight value calculator 134_4. Descriptions of components that operate substantially the same will be omitted.

The time processor 133_4 may merge the interval data ID and the interpolation data PD to generate the merged data MG. The feature weight value calculator 134_2 may analyze the merged data MG through the feed-forward neural network. The recurrent neural network may analyze the merged data MG and generate the feature analysis data XD, based on the weight value Wr1 and the bias br1. The feature weight value calculator 134_4 may calculate the feature weight value AD, by applying the feature analysis data XD and the modeled masking data to the softmax function. As an example, the feature weight value calculator 134_4 may generate the feature weight value AD, by calculating an equation of AD=softmax (tan h (Wm*MD+bm)+tan h (Wx*XD+bx)).

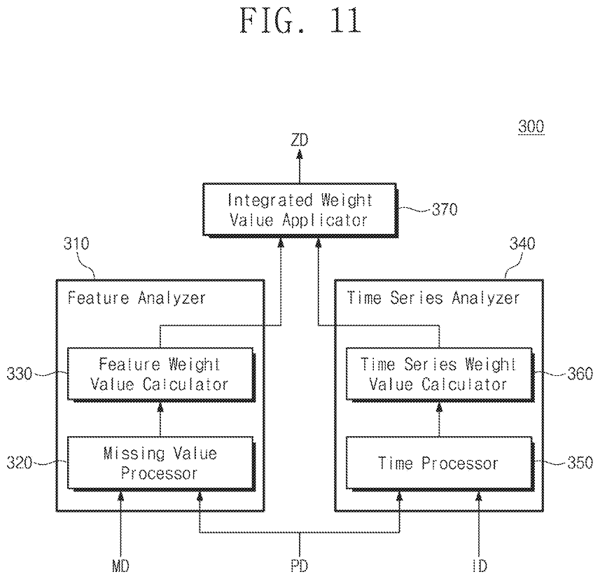

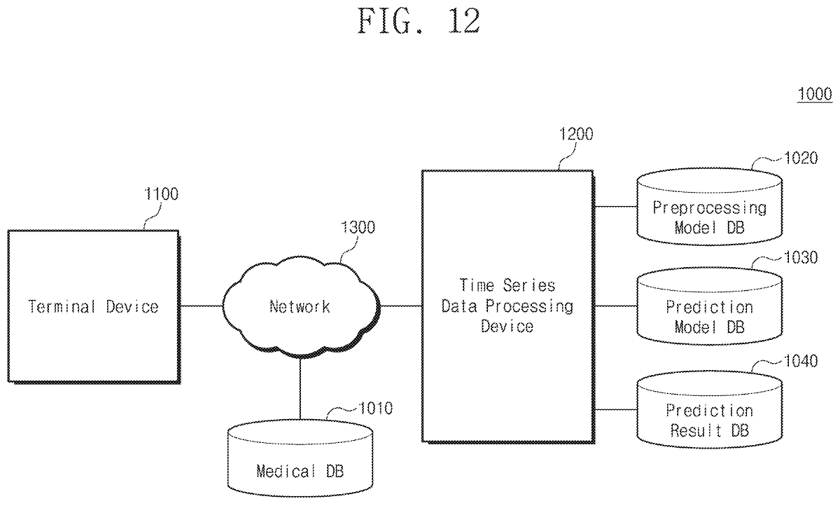

FIGS.10 and 11 are exemplary block diagrams illustrating a learner or a predictor of FIG. 1. An analyzer 200 illustrated in FIG. 10 may be implemented by the learner 120 or the predictor 130 in FIG. 1. Referring to FIG. 10, the analyzer 200 may include a feature analyzer 210 and a time series analyzer 250. As described in FIG. 1, the feature analyzer 210 and the time series analyzer 250 may be implemented in hardware, firmware, software, or a combination thereof.

The feature analyzer 210 analyzes the feature of the time series data, based on the interpolation data PD and the masking data MD. Unlike the feature learner 121 of FIG. 4, the feature analyzer 210 may not use the interval data ID. To this end, a missing value processor 220, a feature weight value calculator 230, and a feature weight value applicator 240 may be implemented in the feature analyzer 210. The missing value processor 220, the feature weight value calculator 230, and the feature weight value applicator 240 may operate substantially the same as the missing value processor 122, the feature weight value calculator 124, and the feature weight value applicator 125, in FIG. 4, except that the interval data ID is not applied to the calculation of the feature weight value.

In detail, the missing value processor 220 may generate the correction data that are obtained by correcting the interpolation value of the interpolation data PD, based on the interpolation data PD and the masking data MD. The feature weight value calculator 230 may calculate the feature weight value corresponding to features and times of the interpolation data PD, based on the correction data. The feature weight value applicator 240 may generate the first result, by applying the calculated feature weight to the interpolation data PD or an intermediate result (the feature analysis data XD of FIGS. 6 to 9) of the interpolation data PD.

The time series analyzer 250 analyzes the time flow of the time series data, based on the first result and the interval data ID of the feature analyzer 210. To this end, a time processor 260, a time series weight value calculator 270, and a time series weight value applicator 280 may be implemented in the time series analyzer 250. Unlike the time series learner 126 of FIG. 4, the time series analyzer 250 may apply the irregularity of the time interval to the time flow analysis, through the time processor 260. The first result may include an error that is generated due to an irregular time interval. The time processor 260 may correct the error, based on the interval data ID.

In detail, the time processor 260 may generate the correction data that are obtained by correcting the first result, based on the interval data ID. This may correspond to the manner in which the time processor 123 of FIG. 4 corrects the interpolation data PD. The time series weight value calculator 270 may calculate the time series weight value corresponding to the plurality of times, based on the correction data. The time series weight value applicator 280 may generate the second result ZD, by applying the calculated time series weight value to the first result or the intermediate result (the time series analysis data HD of FIGS. 6 to 9) of the first result.

When the analyzer 200 is implemented as the learner 120 of FIG. 1, the parameter of the weight value group may be adjusted based on the second result ZD. When the analyzer 200 is implemented as the predictor 130 of FIG. 1, the prediction result corresponding to the prediction time may be generated based on the second result ZD.

FIG. 11 is exemplary block diagrams illustrating a learner or a predictor of FIG. 1. An analyzer 300 illustrated in FIG. 11 may be implemented as the learner 120 or the predictor 130 in FIG. 1. Referring to FIG. 11, the analyzer 300 may include a feature analyzer 310, a time series analyzer 340, and an integrated weight value applicator 370. As described in FIG. 1, the feature analyzer 310, the time series analyzer 340, and the integrated weight value applicator 370 may be implemented in hardware, firmware, software, or a combination thereof.

The feature analyzer 310 analyzes the feature of the time series data and generates the feature weight value, based on the interpolation data PD and the masking data MD. To this end, a missing value processor 320 and a feature weight value calculator 330 may be implemented in the feature analyzer 310. The missing value processor 320 may generate first correction data that are obtained by correcting the interpolation value of the interpolation data PD, based on the interpolation data PD and the masking data MD. The feature weight value calculator 330 may calculate the feature weight value corresponding to the features and the times of the interpolation data PD, based on the first correction data.

The time series analyzer 340 analyzes the time flow of the time series data and generates the time series weight value, based on the interpolation data PD and the interval data ID. To this end, a time processor 350 and a time series weight value calculator 360 may be implemented in the time series analyzer 340. The time processor 350 may generate the second correction data that are obtained by correcting the irregularity of the time interval of the interpolation data PD, based on the interpolation data PD and the interval data ID. The time series weight value calculator 360 may calculate the time series weight value corresponding to the times of the interpolation data PD, based on the second correction data.

The integrated weight value applicator 370 may apply the feature weight value calculated from the feature analyzer 310 and the time series weight value calculated from the time series analyzer 340, to the interpolation data PD. For example, the feature and the time of the time series data may be analyzed in parallel, and the feature weight value and the time series weight value may be applied to the time series data together. As a result of applying the feature weight value and the time series weight value, a result ZD may be generated. When the analyzer 300 is implemented as the learner 120 of FIG. 1, the parameter of the weight value group may be adjusted based on the result ZD. When the analyzer 300 is implemented as the predictor 130 of FIG. 1, the prediction result corresponding to the prediction time may be generated based on the result ZD.

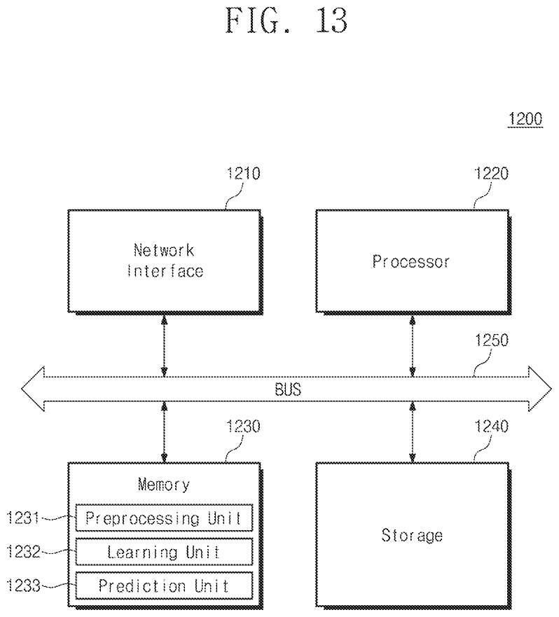

FIG. 12 is a diagram illustrating a health condition prediction system to which a time series data processing device of FIG. 1 is applied. Referring to FIG. 12, the health condition prediction system 1000 includes a terminal device 1100, a time series data processing device 1200, and a network 1300.

The terminal device 1100 may collect the time series data from a user and provide the time series data to the time series data processing device 1200. For example, the terminal device 1100 may collect the time series data from a medical database 1010 or the like. The terminal device 1100 may be one of various electronic devices capable of receiving the time series data from the user, such as a smartphone, a desktop, a laptop, a wearable device, and the like. The terminal device 1100 may include a communication module or a network interface to transmit the time series data through the network 1300. Although the terminal device 1100 is illustrated as one in FIG. 12, the inventive concept is not limited thereto, and the time series data from a plurality of terminal devices may be provided to the time series data processing device 1200.

The medical database 1010 is configured to integrally manage the medical data for various users. The medical database 1010 may include the learning database 101 or the target database 102 of FIG. 1. For example, the medical database 1010 may receive the medical data from public institutions, hospitals, users, or the like. The medical database 1010 may be implemented in a server or a storage medium. The medical data may be managed, grouped, and stored in time series in the medical database 1010. The medical database 1010 may periodically provide the time series data to the time series data processing device 1200 through the network 160.

The time series data may include time series medical data that indicates a user health conditions generated by diagnosis, treatment, or dosage prescription in a medical institution, such as the electronic medical record (EMR). The time series data may be generated when visiting the medical institution for diagnosis, treatment, or dosage prescription. The time series data may be data listed in time series, depending on the visit of the medical institution. The time series data may include a plurality of features that are generated based on the features of diagnosis, treatment, or dosage prescription. For example, the feature may include data measured by a test such as blood pressure or data indicating the extent of a disease such as atherosclerosis.

The time series data processing device 1200 may construct the learning model through the time series data that are received from the medical database 1010 (or the terminal device 1100). For example, the learning model may include a predictive model for predicting future health conditions, based on the time series data. For example, the learning model may include a preprocessing model for preprocessing the time series data. The time series data processing device 1200 may learn the learning model and generate the weight value group, through the time series data that are received from the medical database 1010. To this end, the preprocessor 110 and the learner 120 of FIG. 1 may be implemented in the time series data processing device 1200.

The time series data processing device 1200 may process the time series data that are received from the terminal device 1100 or the medical database 1010, based on the constructed learning model. The time series data processing device 1200 may preprocess the time series data, based on the constructed preprocessing model. The time series data processing device 1200 may analyze the preprocessed time series data, based on the constructed prediction model. As a result of the analysis, the time series data processing device 1200 may calculate the prediction result corresponding to the prediction time. The prediction result may correspond to the future health conditions of the user. To this end, the preprocessor 110 and the predictor 130 of FIG. 1 may be implemented in the time series data processing device 1200.

A preprocessing model database 1020 is configured to integrally manage the preprocessing model and the weight value group that are generated by learning in the time series data processing device 1200. The preprocessing model database 1020 may be implemented in a server or a storage medium. For example, the preprocessing model may include a model for interpolating the missing value for features included in the time series data.

A prediction model database 1030 is configured to integrally manage the prediction model and the weight value group that are generated by learning in the time series data processing device 1200. The prediction model database 1030 may include the weight value model database 103 of FIG. 1. The prediction model database 1030 may be implemented in a server or a storage medium.

A prediction result database 1040 is configured to integrally manage the prediction result that is analyzed in the time series data processing device 1200. The prediction result database 1040 may include the prediction result database 104 of FIG. 1. The prediction result database 1040 may be implemented in a server or a storage medium.

The network 1300 may be configured to perform data communication among the terminal device 1100, the medical database 1010, and the time series data processing device 1200. The terminal device 1100, the medical database 1010, and the time series data processing device 1200 may exchange data by wire or wirelessly through the network 1300.

FIG. 13 is an exemplary block diagram illustrating a time series data processing device of FIG. 1 or FIG. 12. The block diagram of FIG. 13 will be understood as an exemplary configuration for preprocessing the time series data, generating the weight value group, based on the preprocessed time series data, and generating the prediction result, based on the weight value group, and a structure of the time series data processing device will not be limited to thereto. Referring to FIG. 13, the time series data processing device 1200 may include a network interface 1210, a processor 1220, a memory 1230, storage 1240, and a bus 1250. As an example, the time series data processing device 1200 may be implemented as a server, but is not limited thereto.

The network interface 1210 is configured to receive the time series data that are provided from the terminal device 1100 or the medical database 1010 through the network 1300 of FIG. 12. The network interface 1210 may provide the received time series data to the processor 1220, the memory 1230, or the storage 1240 through the bus 1250. In addition, the network interface 1210 may be configured to provide the terminal device 1100 or the like through the network 1300 of FIG. 1 with a prediction result of future health conditions that are generated in response to the received time series data.

The processor 1220 may perform a function as a central processing unit of the time series data processing device 1200. The processor 1220 may perform a control operation and a calculation operation that are required to implement the preprocessing and data analysis of the time series data processing device 1200. For example, under control of the processor 1220, the network interface 1210 may receive the time series data from the outside. Under the control of the processor 1220, the calculation operation for generating the weight value group of the prediction model may be performed, and the prediction result may be calculated using the prediction model. The processor 1220 may operate by utilizing a calculation space, and may read files for driving an operating system and executable files of an application from the storage 1240. The processor 1220 may execute the operating system and various applications.

The memory 1230 may store data and process codes processed by or to be processed by the processor 1220. For example, the memory 1230 may store the time series data, information for performing the preprocessing operation of the time series data, information for generating the weight value group, information for calculating the prediction result, and information for constructing the prediction model. The memory 1230 may be used as a main memory device of the time series data processing device 1200. The memory 1230 may include a dynamic RAM (DRAM), a static RAM (SRAM), a phase-change RAM (PRAM), a magnetic RAM (MRAM), a ferroelectric RAM (FeRAM), a resistive RAM (RRAM), and the like.

A preprocessing unit 1231, a learning unit 1232, and a prediction unit 1233 may be loaded into the memory 1230 and executed. The preprocessing unit 1231, the learning unit 1232, and the prediction unit 1233 correspond to the preprocessor 110, the learner 120, and the predictor 130 of FIG. 1, respectively. The preprocessing unit 1231, the learning unit 1232, and the prediction unit 1233 may be part of the calculation space of the memory 1230. In this case, the preprocessing unit 1231, the learning unit 1232, and the prediction unit 1233 may be implemented by firmware or software. For example, the firmware may be stored in the storage 1240 and loaded into the memory 1230 when executing the firmware. The processor 1220 may execute firmware loaded in the memory 1230. The preprocessing unit 1231 may be operated to preprocess the time series data under the control of the processor 1220. The learning unit 1232 may be operated to analyze the preprocessed time series data to generate the weight value group, under the control of the processor 1220. The prediction unit 1233 may be operated to generate the prediction result, based on the weight value group generated under the control of the processor 1220.

The storage 1240 may store data that are generated for long-term storage by the operating system or applications, files for driving the operating system, executable files of applications, or the like. For example, the storage 1240 may store files for executing the preprocessing unit 1231, the learning unit 1232, and the prediction unit 1233. The storage 1240 may be used as an auxiliary memory of the time series data processing device 1200. The storage 1240 may include a flash memory, a phase-change RAM (PRAM), a magnetic RAM (MRAM), a ferroelectric RAM (FeRAM), a resistive RAM (RRAM), and the like.

The bus 1250 may provide communication paths among components of the time series data processing device 1200. The network interface 1210, the processor 1220, the memory 1230, and the storage 1240 may exchange data from one another through the bus 1250. The bus 1250 may be configured to support various types of communication formats that are used in the time series data processing device 1200.

According to embodiments of the inventive concept, a time series data processing device and an operating method thereof may improve accuracy and reliability of a prediction result, by preprocessing time series data in consideration of irregular time intervals and missing values.

According to embodiments of the inventive concept, a time series data processing device and an operating method thereof may improve accuracy and reliability of the prediction result, by constructing a prediction model that is obtained by comprehensively considering weight values with regard to a time and a feature of the time series data.

The contents described above are specific embodiments for implementing the inventive concept. The inventive concept may include not only the embodiments described above but also embodiments in which a design is simply or easily capable of being changed. In addition, the inventive concept may also include technologies easily changed to be implemented using embodiments. Therefore, the scope of the inventive concept is not limited to the described embodiments but should be defined by the claims and their equivalents.

What is claimed is:

1. A time series data processing device comprising:
a preprocessor configured to generate interval data, based on a time interval of time series data, add an interpolation value to a missing value of the time series data to generate interpolation data, and generate masking data for distinguishing the missing value; and
a learner configured to generate a weight value group of a prediction model that generates a feature weight value depending on a time and a feature of the time series data and a time series weight value depending on a time flow of the time series data, based on the interval data, the interpolation data, and the masking data,
wherein the weight value group includes a first parameter for generating the feature weight value and a second parameter for generating the time series weight value.

2. The time series data processing device of claim 1, wherein the learner includes:
a feature learner configured to calculate the feature weight value, based on the masking data, the interval data, the interpolation data, and the first parameter, and generate a first learning result, based on the feature weight value;
a time series learner configured to calculate the time series weight value, based on the first learning result and the second parameter, and generate a second learning result, based on the time series weight value; and
a weight value controller configured to adjust the first parameter or the second parameter, based on the first learning result or the second learning result.

3. The time series data processing device of claim 2, wherein the feature learner includes:
a missing value processor configured to generate first correction data of the interpolation data, based on the masking data;
a time processor configured to generate second correction data of the interpolation data, based on the interval data;
a feature weight value calculator configured to calculate the feature weight value, based on the first parameter, the first correction data, and the second correction data; and
a feature weight value applicator configured to apply the feature weight value to the interpolation data.

4. The time series data processing device of claim 2, wherein the time series learner includes:
a time series weight value calculator configured to calculate the time series weight value, based on the first learning result and the second parameter; and
a time series weight value applicator configured to apply the time series weight value to the first learning result.

5. The time series data processing device of claim 1, wherein the learner includes:
a feature learner configured to calculate the feature weight value, based on the masking data, the interpolation data, and the first parameter, and generate a first learning result, based on the feature weight value;
a time series learner configured to calculate the time series weight value, based on the interval data, the first learning result, and the second parameter, and generate a second learning result, based on the time series weight value; and
a weight value controller configured to adjust the first parameter or the second parameter, based on the first learning result or the second learning result.

6. The time series data processing device of claim 5, wherein the feature learner includes:
a missing value processor configured to generate correction data of the interpolation data, based on the masking data;
a feature weight value calculator configured to calculate the feature weight value, based on the first parameter and the correction data; and
a feature weight value applicator configured to apply the feature weight value to the interpolation data.

7. The time series data processing device of claim 5, wherein the time series learner includes:
a time processor configured to generate correction data of the first learning result, based on the interval data;
a time series weight value calculator configured to calculate the time series weight value, based on the second parameter and the correction data; and
a time series weight value applicator configured to apply the time series weight value to the first learning result.

8. The time series data processing device of claim 1, wherein the learner includes:
a feature learner configured to calculate the feature weight value, based on the masking data, the interpolation data, and the first parameter;
a time series learner configured to calculate the time series weight value, based on the interval data, the interpolation data, and the second parameter; and
an integrated weight value applicator configured to generate a learning result, based on the feature weight value and the time series weight value; and
a weight value controller configured to adjust the first parameter or the second parameter, based on the learning result.

9. A time series data processing device comprising:
a preprocessor configured to generate interval data, based on a time interval of time series data, add an interpolation value to a missing value of the time series data to generate interpolation data, and generate masking data for distinguishing the missing value; and
a predictor configured to generate a feature weight value depending on a time and a feature of the time series data and a time series weight value depending on a time flow of the time series data, based on the interval data, the interpolation data, and the masking data, and generate a prediction result, based on the feature weight value and the time series weight value.

10. The time series data processing device of claim 9, wherein the predictor includes:
a feature predictor configured to generate a first result, based on the feature weight value;
a time series predictor configured to generate a second result, based on the time series weight value; and
a result generator configured to calculate the prediction result corresponding to a prediction time, based on the second result.

11. The time series data processing device of claim 10, wherein the feature predictor includes:
a missing value processor configured to encode the interpolation data, based on the masking data;
a time processor configured to model the interval data;
a feature weight value calculator configured to generate feature analysis data, based on the encoded interpolation data and to generate the feature weight value, based on the feature analysis data and the modeled interval data; and
a feature weight value applicator configured to apply the feature weight value to the feature analysis data to generate the first result.

12. The time series data processing device of claim 10, wherein the feature predictor includes:
a missing value processor configured to merge the masking data and the interpolation data;
a time processor configured to model the interval data;
a feature weight value calculator configured to generate feature analysis data, based on the merged data, and generate the feature weight value, based on the feature analysis data and the modeled interval data; and
a feature weight value applicator configured to apply the feature weight value to the feature analysis data to generate the first result.

13. The time series data processing device of claim 10, wherein the feature predictor includes:
a missing value processor configured to model the masking data;
a time processor configured to model the interval data;
a feature weight value calculator configured to generate feature analysis data, based on the interpolation data, and generate the feature weight value, based on the modeled masking data, the modeled interval data, and the feature analysis data; and
a feature weight value applicator configured to apply the feature weight value to the feature analysis data to generate the first result.

14. The time series data processing device of claim 10, wherein the feature predictor includes:
a missing value processor configured to model the masking data;
a time processor configured to merge the interval data and the interpolation data;
a feature weight value calculator configured to generate feature analysis data, based on the merged data, and generate the feature weight value, based on the feature analysis data and the modeled masking data; and
a feature weight value applicator configured to apply the feature weight value to the feature analysis data to generate the first result.

15. The time series data processing device of claim 10, wherein the time series predictor includes:
a time series weight value calculator configured to generate time series analysis data, based on the first result, and generate the time series weight value, based on the time series analysis data; and
a time series weight value applicator configured to apply the time series weight value to the first result or the time series analysis data.

16. The time series data processing device of claim 10, wherein the feature predictor calculates the feature weight value, based on the masking data and the interpolation data, and
wherein the time series predictor calculates the time series weight value, based on the first result and the interval data.

17. The time series data processing device of claim 9, wherein the predictor includes:
a feature predictor configured to calculate the feature weight value, based on the masking data and the interpolation data;
a time series predictor configured to calculate the time series weight value, based on the interval data the interpolation data;
an integrated weight value applicator configured to generate an integrated result corresponding to the interpolation data, based on the feature weight value and the time series weight value; and
a result generator configured to calculate the prediction result corresponding to a prediction time, based on the integrated result.

18. A method of operating a time series data processing device, the method comprising:
generating interpolation data by adding an interpolation value to a missing value of time series data;
generating interval data, based on a time interval of the time series data;
generating masking data, based on the missing value;
generating a feature weight value depending on a time and a feature of the time series data, based on the interpolation data, the interval data, and the masking data;
generating a first result, based on the feature weight value;
generating a time series weight value depending on a time flow of the time series data, based on the first result; and
generating a second result, based on the time series weight value.

19. The method of claim 18, further comprising:
adjusting a parameter for generating the feature weight value or the time series weight value, based on the second result.

20. The method of claim 18, further comprising:
calculating a prediction result corresponding to a prediction time, based on the second result.
